(19) United States
(12) Patent Application Publication
(10) Pub. No.: US 2012/0202843 A1
(43) Pub. Date: Aug. 9, 2012

(54) H3K27me3 and Cancer

(75) Inventors: Karl Munger, Newton, MA (US); Christopher Crum, Brookline, MA (US); Margaret McLaughlin-Drubin, Jamaica Plain, MA (US)

(73) Assignee: THE BRIGHAM AND WOMEN'S HOSPITAL, INC., Boston, MA (US)

(21) Appl. No.: 13/383,106

(22) PCT Filed: Jul. 14, 2010
(86) PCT No.: PCT/US10/41962

§ 371 Date: Apr. 25, 2012

Related U.S. Application Data
(60) Provisional application No. 61/225,645, filed on Jul. 15, 2009.

(51) Int. Cl. A61K 31/4745; A61P 35/00; C12Q 1/68; G01N 33/574; G01N 33/573; G01N 33/566

(52) U.S. Cl. 514/293; 435/7.4; 435/7.1; 435/6.12

(57) ABSTRACT

Methods of diagnosing and monitoring cancers and precancerous lesions associated with human papillomavirus (HPV), by detecting abnormal levels of lysine (K)-specific demethylase 6A (KDM6A), KDM6B, or trimethylated Histone H3 Lys27 (H3K27me3).

### CLAIM OF PRIORITY

This application claims the benefit of U.S. Patent Application Ser. No. 61/225,645, filed on Jul. 15, 2009, the entire contents of which are hereby incorporated by reference.

### FEDERALLY SPONSORED RESEARCH OR DEVELOPMENT

This invention was made with Government support under Grant Nos. CA066980 and K12 HD051959 awarded by the Public Health Service of the National Institutes of Health. The Government has certain rights in the invention.

### TECHNICAL FIELD

This invention relates to methods of diagnosing and monitoring cancers and precancerous lesions associated with human papillomavirus (HPV), by detecting abnormal levels of lysine (K)-specific demethylase 6A (KDM6A), KDM6B, or Histone H3 Lys27 trimethyl (H3K27me3).

### BACKGROUND

Cervical cancer is the second most common cancer among women worldwide and is the most common female cancer in developing countries (Mohar and Frias-Mendivil, 2000. Invest 18:584-590; Parkin et al., 1999. CA Cancer J Clin 49:33-64, 1). In the United States, cervical cancer mortality is highest in medically underserved populations (Schwartz et al., 2003. Cancer Causes Control 14:761-6). The most significant risk factor in its etiology is infection with high-risk human papillomavirus (HPV) types (Cancer, I. A. f. R. o. 1995. IARC monographs on the evaluation of carcinogenic risks to humans, vol. Vol. 63. Sons Presse, Lyon [France]); in fact, over 99% of all cervical cancers examined have been associated with high-risk HPVs (Walboomers et al., 1999. J Pathol 189:12-9).

Although preventative HPV vaccination strategies appear promising (Koutsky et al., 2002. N Engl J Med 347:1645-51), it will be decades before an impact on the incidence of HPV-associated disease and mortality is seen (Frazer, 2004. Nat Rev Immunol 4:46-54). Therefore, continued population-wide screening is crucial for the identification of asymptomatic women with preneoplastic lesions or early cancers of the cervix (Kurman et al., 1994. JAMA 271:1866-9). Despite the success of programs based on cytological screening using the Papanicolaou's smear test (Pap test), Pap testing is affected by a substantial number of false-negative and false-positive test results (Anderson et al., 1988. 1955-85. Br Med J (Clin Res Ed) 296:975-8; Hakama, 1996. Cancer Treat Res 86:41-9; Laara et al., 1987. Lancet 1:1247-9; Papanicolaou, 1942. Science 95:438-439), causing repeated testing, an unnecessary emotional burden to women, and great additional health-related costs (Follen and Richards-Kortum, 2000. J Natl Cancer Inst 92:363-5; Kurman et al., 1994. JAMA 271:1866-9; Schechter, 1996. Acta Cytol 40:1272-82). Moreover, histological analysis of biopsy samples from women with abnormal Pap smears is also subject to a high rate of intraobserver and interobserver diagnostic variability (de Vet et al., 1990. J Clin Epidemiol 43:1395-8; Robertson et al., 1989. J Clin Pathol 42:231-8), indicating the need for objective biomarkers.

To date, only one biomarker for the identification of dysplastic cervical cells is used in the clinic, p16^{INK4A} (Klaes et al., 2001. Int J Cancer 92:276-284; Sano et al., 1998. Am J Pathol 153:1741-8). Although in the initial report by Sano et al. staining for p16 was strongly associated with HPV16-associated squamous intraepithelial lesions, the association between p16 staining and high-risk HPVs was not absolute in other studies (Keating et al., 2001. Am J Surg Pathol 25:884-91). Furthermore, simply detecting the presence of HPV in a sample of cells (e.g., using PCR-based detection assays) can lead to false-positives as well, since transient and non-pathogenic infections would also be detected using this method (Kulasingam, JAMA. 2002; 288(14):1749-57) Therefore, there is still a significant demand for biomarkers to identify dysplastic or neoplastic cervical epithelial cells.

### SUMMARY

The present invention is based, at least in part, on the discovery that KDM6A, KDM6B, and/or H3K27me3 are differentially expressed in cells from HPV-associated precancerous lesions or cancers as compared to normal cells, and that levels of KDM6A, KDM6B, and/or H3K27me3 return to normal levels after the transformative stimulus is removed. Thus, described herein are methods for diagnosing an HPV-associated precancerous lesion or cancer or risk of developing an HPV-associated precancerous lesion or cancer, as well as monitoring therapeutic efficacy in HPV-associated precancerous lesions or cancers.

In one aspect, the invention provides methods for diagnosing a human papilloma virus (HPV)-associated cancer or precancerous lesion in a subject. The methods include obtaining a sample comprising a cell, e.g., a cell suspected of being from an HPV-associated cancer or precancerous lesion, from the subject; and evaluating the presence and/or level of one or more biomarkers selected from the group consisting of lysine (K)-specific demethylase 6A (KDM6A), KDM6B, or Histone H3 Lys27 trimethyl (H3K27me3) in the sample; wherein the presence and/or level of the one or more biomarkers indicates whether the subject has an HPV-associated cancer or precancerous lesion.

In some embodiments, the methods further include comparing the level of the biomarker with a reference level, wherein the comparison of the level of the biomarker with the reference indicates whether the subject has an HPV-associated cancer or precancerous lesion. In some embodiments, the reference is a control reference that represents a level of KDM6A, KDM6B, and/or H3K27me3 in a normal cell, or a disease reference that represents a level of KDM6A, KDM6B, and/or H3K27me3 in a cell from an HPV-associated cancer or precancerous lesion.

In some embodiments, the level of H3K27me3 is determined, and the level of H3K27me3 is reduced as compared to a level in a normal control cell, then the subject is diagnosed with an HPV-associated cancerous or precancerous cell.

In some embodiments, the presence of H3K27me3 is determined, and the absence of detectable levels of H3k27me3 indicates that the subject has an HPV-associated cancer or precancerous lesion.

In some embodiments, the level of H3K27me3 is determined, a level of total histone3 (H3) is determined, and a ratio of H3K27me3 to total H3 is calculated, wherein the ratio of H3K27me3 to total H3 in the cell indicates whether the cell is from an HPV-associated cancer or precancerous lesion. In some embodiments, the ratio is compared to a ratio in a reference cell, e.g., a normal or disease cell, and the comparison of the ratio to the reference ratio indicates whether the sample is or includes precancerous or cancerous cells.

In some embodiments, a level of KDM6A or KDM6B is determined, and the presence of a level of KDM6A or KDM6B that is be significantly increased as compared to a normal control cell indicates that the subject has an HPV-associated cancer or precancerous lesion.

In some embodiments, the methods include selecting and/or administering a treatment for an HPV-associated cancer or precancerous lesion to the subject.

In another aspect, the invention provides methods for monitoring the efficacy of a treatment for an HPV-associated cancer or precancerous lesion. The methods include obtaining a first sample from a subject who has (or is suspected of having) an HPV-associated cancer and/or precancerous lesion at a first time point, and evaluating the presence and/or level of one or more of KDM6A, KDM6B, and/or H3K27me3 in the first sample; administering a treatment to the subject; obtaining a second sample from the subject at a subsequent time point, and evaluating the presence and/or level of one or more of KDM6A, KDM6B, and/or H3K27me3 in the second sample; comparing the level of the KDM6A, KDM6B, and/or H3K27me3 in the first and second sample, wherein a decrease in the level of KDM6A, KDM6B, and/or an increase in the level of H3K27me3 between the first and second samples indicates that the treatment is effective. A similar method can be used to monitor the progress of a lesion, e.g., in a subject who has or has had an HPV infection. The methods include determining a first or baseline level of the biomarker(s), and at a subsequent time point determining a second or additional level; no change or a decrease in the level of the biomarker indicates that there is no disease progression, while an increase in the levels would indicate that there is disease progression.

In some embodiments, the methods described herein include determining a level of KDM6A, KDM6B, and/or H3K27me3 protein or mRNA.

In some embodiments, the subject is a mammal, e.g., a human.

In some embodiments, the HPV-associated cancer or precancerous lesion is in a tissue selected from the group consisting of cervix, vulva, vagina, penis, anus, oral cavity, oropharynx, breast, skin, prostate, and lung.

In some embodiments, the methods include determining a level of an additional marker of HPV-associated cancer, e.g., p16^{INK4A}, or an HPV viral nucleic acid or protein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-B show that the repressive H3K27me3 mark is specifically reduced in HPV16 E7 expressing primary human epithelial cells. FIG. 1A is a set of six images showing the results of immunofluorescence analysis of histone H3 methylation in primary human epithelial cells (HFKs) and donor and passage-matched HPV16 E7 expressing HFKs (HFK/E7). FIG. 1B is an image showing the results of Western blot analysis of H3K27me3 levels in HFKs and HFK/E7s. Lysates were separated by SDS-PAGE, transferred, and probed for H3K27me3 (me3) and total H3 as a loading control.

FIGS. 2A-C show that increased expression of KDM6A and KDM6B in HPV16 E7 expressing primary human epithelial cells. FIG. 2A is a set of four images showing the results of immunofluorescence analysis of KDM6B (upper panels) and KDM6A (lower panels) expression in donor and passage matched populations of primary human foreskin keratinocytes (HFKs) (left panels) and HPV16 E7 expressing HFKs (HFK/E7) (right panels). FIG. 2B is an image showing the results of Western blot analysis of KDM6A and KDM6B levels in HFKs and HFK/E7 cells. Lysates were separated by SDS-PAGE, transferred, and probed for KDM6A, KDM6B and HPV16 E7. A pRB blot is shown to document functional E7 expression and an actin blot is included as a loading control. FIG. 2C is a bar graph showing the results of quantitative real-time RT-PCR analysis of KDM6A and KDM6B mRNA expression in HFK and HFK/E7 cells. The bar graph shows averages and standard deviations from 3 independent experiments each performed in triplicate. Increases in HFK E7 cells are statistically significant (*) with p values <0.005.

FIGS. 3A-D show that HPV16 E7-mediated induction of KDM6B is critical for the expression of the cervical cancer biomarker p16^{INK4A}. FIGS. 3A-D are each four images showing the results of co-immunofluorescence staining of p16^{INK4A} and H3K27me3 in rafts prepared from (3A) primary human foreskin keratinocytes, (3B) HPV16 immortalized human foreskin keratinocytes, and (3C) in an HPV16 positive cervical intraepithelial neoplasia (CIN) specimen. FIG. 3C top and bottom rows are from different areas of the same specimen. Similar staining patterns were detected in three additional CIN specimens. Hoechst staining was used to visualize nuclei and a phase picture (left column) shows cellular morphology. FIG. 3D is an image of a Western blot (left) and a bar graph (right) showing the results of experiments in which monolayer cultures of HPV16-immortalized HFKs were transfected with KDM6B specific siRNA duplexes or control siRNA (CTL), and p16^{INK4A} and p14^{ARF} expression was analyzed by Western blotting at 72 hours post transfection. An actin blot is shown as a loading control (bottom panel). Averages and standard deviations of KDM6B, p16^{INK4A} and p14^{ARF} levels from 3 independent experiments are shown on the bar graph in the right panel. Statistically significant decreases (p<0.05) are indicated by a (*).

FIG. 4 shows that HPV16 E7-mediated induction of KDM6B and its transcriptional target p16^{INK4A} are not dependent on the ability of HPV16 E7 to degrade pRB. The image shows the results of Western blot analysis of KDM6B and p16^{INK4A} expression in donor and passage matched populations of primary human fibroblasts (HFFs) expressing wild type HPV16 E7 (WT), the pRB binding/degradation deficient HPV16 delD21-24 mutant, and control vector infected HFFs. Lysates were separated by SDS-PAGE, transferred, and probed for KDM6B, p16^{INK4A}, and HPV16 E7. An actin blot is show as a loading control.

FIG. 5 is a bar graph showing dysregulation of HOX gene expression in HPV16 E7 expressing primary human epithelial cells. Quantitative real-time RT-PCR of HOX mRNA expression in primary human foreskin keratinocytes (HFKs) and donor and passage-matched HPV16 E7 expressing HFKs (HFK/E7). Bar graphs represent averages and standard deviations of 3 experiments each performed in triplicate. HOX genes that exhibit significant (p<0.05) upregulation are marked (*). While all 39 genes in the HOX A-D clusters were analyzed, those HOX genes that were expressed in HFKs at levels too low to evaluate (HOXA7, All, A13, B1, B3, B5, B6, B8, B9, B13, D1, D3, D4, D9, D10, D11, D12, and D13) are not shown.

FIGS. 6A-B show that HPV16 E7-mediated induction of KDM6B and p16^{INK4A} and decreases in H3K27me3 levels are reversible. FIG. 6A is an image showing the results of Western blot analysis of KDM6B, p16^{INK4A}, and HPV16 E7 expression in U2OS-tet on cells with doxycycline-inducible expression of HPV16 E7 treated with doxycycline as indicated. Lysates were separated by SDS-PAGE, transferred, and probed with the indicated antibodies. An actin blot is show as a loading control. FIG. 6B is a set of eight images showing the results of immunofluorescence analysis of histone H3 lysine 27 trimethylation in U2OS-tet on cells with doxycycline-inducible expression of HPV16 E7 treated with doxycycline as indicated. Hoechst stain was used to visualize nuclei.

FIG. 7 is a schematic hypothetical model for epigenetic reprogramming by the HPV16 E7 oncoprotein.

### DETAILED DESCRIPTION

Human Papillomaviruses (HPVs) are small double-stranded DNA viruses that are associated with epithelial hyperplasias. A subgroup of “high-risk” HPVs are etiologic agents of cervical carcinomas as well as other anogenital cancers and some oropharyngeal tumors (Schiffman et al., (2007) Lancet 370(9590):890-907). Due to frequent integration of the viral genome into a host cell chromosome, E6 and E7 are the only viral proteins that are consistently expressed in HPV associated cancers. E6 and E7 have oncogenic activities, and their expression is necessary for the induction and maintenance of the transformed phenotype (McLaughlin-Drubin and Munger (2009) Virus Res 143(2):195-208).

The transforming activities of E6 and E7 are a reflection of their functions during the viral life cycle. HPVs initially infect the proliferating basal cells of a squamous epithelium. Production of infectious progeny, however, is restricted to terminally differentiated layers of the infected epithelium, and the HPV E6 and E7 proteins function to retain these cells in a replication competent state (McLaughlin-Drubin and Munger (2009) Virus Res 143(2):195-208).

The HPV E6 and E7 proteins lack intrinsic enzymatic activities and do not act as DNA binding transcription factors; rather, they reprogram their host cells by associating with cellular signaling molecules, including transcription factor complexes. High-risk HPV E6 proteins target the p53 tumor suppressor for degradation, thereby thwarting p53-mediated transcriptional cytostatic and cytotoxic responses to cellular stress signals. High-risk HPV E7 oncoproteins associate with and degrade the retinoblastoma tumor suppressor (pRB), which acts as a cell cycle specific repressive subunit of several E2F transcriptional complexes, and thereby subvert cell cycle dependent E2F transcriptional activities (McLaughlin-Drubin and Munger (2009) Virus Res 143(2):195-208). The HPV E6 and E7 oncoproteins also associate with enzymes that modulate histone acetylation, and thus broadly regulate the transcriptional competence of host cell chromatin (Brehm et al. (1999) Embo J 18(9):2449-2458; Longworth and Laimins (2004) J Virol 78(7):3533-3541; Avvakumov et al., (2003) Oncogene 22(25):3833-3841; Baldwin et al., (2006) J Virol 80(13):6669-6677; Bernat et al., (2003) Oncogene 22(39):7871-7881; Huang and McCance (2002) J Virol 76(17):8710-8721).

The HPV16 E7 oncoprotein associates with E2F6-containing polycomb transcriptional repressor complexes (PRCs) and that the detection of these complexes is reduced in HPV16 E7 expressing cells (McLaughlin-Drubin et al., (2008) J Virol 82(17):8695-8705). PRCs require the histone H3 lysine 27 trimethyl (H3K27me3) mark to associate with and transcriptionally silence chromatin (Schwartz and Pirrotta (2007) Nat Rev Genet 8(1):9-22). PcG proteins have been most extensively studied in Drosophila melanogaster (Shah and Sukumar (2010) Rev Cancer 10(5):361-371) where they establish and sustain stable epigenetic silencing of Homeobox (HOX) genes during development (Gould (1997) Curr Opin Genet Dev 7(4):488-494; Schumacher and Magnuson (1997) Trends Genet 13(5):167-170). HOX proteins are master regulators of transcriptional programs that create and maintain cellular identities. Other PRC regulated genes include the INK4A tumor suppressor locus, which encodes p16^{INK4A}, an inhibitor of cyclin dependent kinases 4 and 6, and p14^{ARF}, an inhibitor of mdm2-mediated p53 degradation (Serrano et al., (1997) Cell 88(5):593-602). Silencing through H3K27me3 involves two distinct complexes, PRC2 and PRC1. The catalytic subunit of PRC2, EZH2, is a methyl transferase that catalyzes di- and trimethylation of H3K27, which is then recognized by PRC1 (Kouzarides (2007) Cell 128(4):693-705). The repressive H3K27me3 mark can be removed by one of two known histone demethylases KDM6A (UTX) and KDM6B (JMJD3). KDM6B has been shown to remove H3K27me3 marks from the p16^{INK4A} promoter during ras/raf oncogene induced cellular senescence (Agger et al. (2009). Genes Dev 23(10):1171-1176; Barradas et al. (2009) Genes Dev 23(10):1177-1182), while KDM6A has been implicated in the removal of H3K27me3 marks from the promoters of several genes encoding RB-binding proteins (Wang et al. (2010) Genes Dev 24(4):327-332).

As described herein, HPV16 E7 expression results in a dramatic reduction of the H3K27me3 mark necessary for the binding of PRC1 through transcriptional induction of the histone demethylases KDM6A and KDM6B. Increased expression of the cervical carcinoma biomarker p16^{INK4A} in HPV16 E7 expressing cells is specifically linked to KDM6B induction. Induction of KDM6B and its transcriptional target p16^{INK4A} is not dependent on pRB inactivation and, hence is not linked to E2F activation by E7. Since HPV16 E7 also inactivates pRB, the mediator of p16^{INK4A} induced senescence, HPV16 E7 expressing cells continue to proliferate despite high p16^{INK4A} levels and several known KDM6A or KDM6B regulated HOX genes are expressed at higher levels in such cells. Hence, HPV16 E7 expression causes epigenetic reprogramming of host cells at the level of histone methylation. Since these HPV16 E7 induced alterations in H3K27me3 levels and associated transcriptional changes are rapidly reversible upon silencing of E7 expression, these results suggest that KDM6A and KDM6B may be targets for therapy of HPV associated lesions and cancers.

Biomarkers

The present methods include detecting the levels of one or more biomarkers selected from the group consisting of lysine (K)-specific demethylase 6A (KDM6A), lysine (K)-specific demethylase 6B (KDM6B), and/or trimethylated histone H3 Lys 27 (H3K27me3).

KDM6A

The KDM6A protein catalyzes the demethylation of tri/dimethylated histone H3; functions in development and tumor suppression (Wang et al., Genes Dev. 24(4):327-32 (2010)). KDM6A is also known as UTX; MGC141941; bA386N14.2; and DKFZp686A03225. The human nucleic acid sequence can be found in the GenBank database at Ref. No. NM_{—}021140.2; the amino acid sequence is at Ref. No. NP_{—}066963.2. Antibodies for the detection of KDM6A and methods for making them are known in the art. For example, commercially available antibodies can be obtained, e.g., from Abcam (Cambridge, Mass.); Millipore, Sigma-Aldrich, R&D Systems, Cell Signaling Technology, OriGene, Novus Biologicals, and/or Epitomics.

KDM6B

Expression of the H3K27 demethylase JMJD3 is induced upon activation of the RAS-RAF signaling pathway (see Agger et al., Gens Dev. 23(10)1171-76 (2009)). KDM6B is also known as JMJD3; KIAA0346. The human nucleic acid sequence can be found in the GenBank database at Ref. No. NM_{—}001080424.1; the amino acid sequence is at Ref. No. NP_{—}001073893.1. Antibodies for the detection of KDM6B and methods for making them are known in the art. For example, commercially available antibodies can be obtained, e.g., from Abcam (Cambridge, Mass.); Millipore, Sigma-Aldrich, R&D Systems, Cell Signaling Technology, OriGene, Novus Biologicals, and/or Epitomics.

Histone 3 and H3K27me3

The nucleosome is the smallest subunit of chromatin and includes approximately 146-147 base pairs of DNA wrapped around an octamer of core histone proteins (two each of H2A, H2B, H3, and H4). Trimethylation of histone H3 on Lys 27 (H3K27me3) is key for cell fate regulation.

Mammalian cells have three known sequence variants of histone H3 proteins, denoted H3.1, H3.2 and H3.3, that are highly conserved differing in sequence by only a few amino acids. The sequences are as follows:

| | | | |
|---|---|---|---|
| MARTKQTARKSTGGKAPRKQLATKAARKSAPATGGVKKPHRYRPGTVALREIRRYQKSTE | 60 | Histone H3.1 | (SEQ ID NO: 1) |
| MARTKQTARKSTGGKAPRKQLATKAARKSAPATGGVKKPHRYRPGTVALREIRRYQKSTE | 60 | Histone H3.2 | (SEQ ID NO: 2) |
| MARTKQTARKSTGGKAPRKQLATKAARKSAPSTGGVKKPHRYRPGTVALREIRRYQKSTE | 60 | Histone H3.3 | (SEQ ID NO: 3) |
| MARTKQTARKSTGGKAPRKQLATKAARKSAP+TGGVKKPHRYRPGTVALREIRRYQKSTE | Con | Histone H3 | (SEQ ID NO: 4) |
| LLIRKLPFQRLVREIAQDFKTDLRFQSSAVMALQEACEAYLVGLFEDTNLCAIHAKRVTI | 120 | | |
| LLIRKLPFQRLVREIAQDFKTDLRFQSSAVMALQEASEAYLVGLFEDTNLCAIHAKRVTI | 120 | | |
| LLIRKLPFQRLVREIAQDFKTDLRFQSAAIGALQEASEAYLVGLFEDTNLCAIHAKRVTI | 120 | | |
| LLIRKLPFQRLVREIAQDFKTDLRFQS+A+ ALQEA EAYLVGLFEDTNLCAIHAKRVTI | Con | | |
| MPKDIQLARRIRGERA | 136 | | |
| MPKDIQLARRIRGERA | 136 | | |
| MPKDIQLARRIRGERA | 136 | | |
| MPKDIQLARRIRGERA | Consensus | | |

These amino acid sequences include a methionine as residue No. 1 that is cleaved off when the protein is processed, hence what is Lysine 28 in the amino acid sequences above (in bold) corresponds to lysine (K) 27.

These three protein variants are encoded by at least fifteen different genes/transcripts, as shown in Table 1.

TABLE 1
Histone 3 Genes

| Histone 3 | | GenBank Id. Nucleic | GenBank Id. Amino |
|---|---|---|---|
| variant | Gene | Acid | Acid |
| H3.1 | HIST1H3A | NM_003529.2 | NP_003520.1 |
| | HIST1H3B | NM_003537.3 | NP_003528.1 |
| | HIST1H3C | NM_003531.2 | NP_003522.1 |
| | HIST1H3D | NM_003530.3 | NP_003521.2 |
| | HIST1H3E | NM_003532.2 | NP_003523.1 |
| | HIST1H3F | NM_021018.2 | NP_066298.1 |
| | HIST1H3G | NM_003534.2 | NP_003525.1 |
| | HIST1H3H | NM_003536.2 | NP_003527.1 |
| | HIST1H3I | NM_003533.2 | NP_003524.1 |
| | HIST1H3J | NM_003535.2 | NP_003526.1 |
| H3.2 | HIST2H3A | NM_001005464.2 | NP_001005464.1 |
| | HIST2H3C | NM_021059.2 | NP_066403.2 |
| | HIST2H3D | NM_001123375.1 | NP_001116847.1 |
| H3.3 | H3F3A | NM_002107.3 | NP_002098.1 |
| | H3F3B | NM_005324.3 | NP_005315.1 |

Antibodies for the detection of H3K27me3 and methods for making them are known in the art. For example, commercially available antibodies can be obtained, e.g., from Abcam, Cell Signaling, SABiosciences, ActiveMotif, and diagenode. Antibodies for the detection of other forms of H3 can also be made or obtained using methods known in the art or obtained commercially from the same or other sources.

Additional Markers of Cancer/Precancerous Lesions

In some embodiments, the methods further include the evaluation of additional markers of cancerous or precancerous lesions. For example, the methods can include the evaluation of p16^{INK4A} (see, e.g., Klaes et al., 2001. Int J Cancer 92:276-284; Sano et al., 1998. Am J Pathol 153:1741-8; and Keating et al., 2001. Am J Surg Pathol 25:884-91; Benevolo et al., Modern Pathology, 2006. 19:384-341). Levels or the presence of HPV viral particles, viral DNA genomes, viral mRNAs or viral proteins can also be detected (see, e.g., Zaravinos, 2009. Int J Biol Markers 24(4) 215-22).

Methods of Diagnosis and Monitoring

The methods described herein can be used to diagnose or determine risk of developing HPV-associated cancers and precancerous lesions. The methods include obtaining a sample from a subject, and evaluating the presence and/or level of one or more of KDM6A, KDM6B, and/or H3K27me3 in the sample, and comparing the presence and/or level with one or more references, e.g., a control reference that represents a normal level of KDM6A, KDM6B, and/or H3K27me3, e.g., a level in a normal (i.e., non-cancerous, non-precancerous) cell (e.g., from the same subject or a control subject), and/or a disease reference that represents a level of KDM6A, KDM6B, and/or H3K27me3 that is associated with cancer or a precancerous lesion, e.g., a level in a cell from an HPV-associated cancer or precancerous lesion. For example, a level of H3K27me3 in a cancerous or precancerous cell may be significantly (i.e., statistically significantly) reduced as compared to a normal control cell, e.g., substantially undetectable, and a level of KDM6A or KDM6B in a cancerous or precancerous cell may be significantly (i.e., statistically significantly) increased as compared to a normal control cell. The methods can include determining levels of KDM6A, KDM6B, and/or H3K27me3 protein or mRNA.

In some embodiments where H3K27me3 is measured, a level of another form or H3 can also be measured, e.g., the non-methylated, mono-methylated, or dimethylated form, or total H3, and a ratio can be calculated. The ratio can be compared to a reference ratio, e.g., a control reference ratio that represents the ratio in a normal, non-cancerous or non-precancerous cell, or a disease reference that represents the ratio in a cancerous or precancerous cell. In some embodiments the ratio in disease (or suspected disease) tissue is compared to normal tissue; for example, a ratio of H3:H3K27me3 in normal tissue is compared to the same ratio in diseased tissue. Based on IF or IHC, the ratio should increase significantly in diseased tissue. In some embodiments, the ratio of H3:H3k27me3 in normal cells or tissue is normalized to 1, and the presence of a ratio above 1 indicates the presence of cancerous or precancerous cells or tissue. In some embodiments, presence of a normalized ratio above 2, 3, 4, 5, or higher, or in a range of about 2-10, 3-10, 4-10, or 5-10 indicates the presence of disease.

The presence and/or level of a protein or mRNA can be evaluated using methods known in the art, e.g., protein levels can be determined using quantitative immunoassay methods, e.g., immunohistochemistry or immunofluorescence, and mRNA levels can be determined using quantitative PCR or northern blot analysis. In some embodiments, high throughput methods, e.g., protein or gene chips as are known in the art (see, e.g., Ch. 12, Genomics, in Griffiths et al., Eds. Modern genetic Analysis, 1999, W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of KDM6A, KDM6B, and/or H3K27me3.

In some embodiments, the presence and/or level of KDM6A, KDM6B, and/or H3K27me3 is comparable to the presence and/or level of the protein(s) in the disease reference, and the subject has one or more symptoms associated with an HPV-associated precancerous lesion or cancer, or the presence of morphologically cancerous or precancerous cells, then the subject is diagnosed with cancer or a precancerous lesion. In some embodiments, the subject has no overt signs or symptoms of an HPV-associated precancerous lesion or cancer, but the presence and/or level of one or more of the proteins evaluated is comparable to the presence and/or level of the protein(s) in the disease reference, and no morphologically cancerous or precancerous cells, then the subject has an increased risk of developing an HPV-associated precancerous lesion or cancer.

In some embodiments, other markers or biomarkers of HPV are also evaluated, e.g., p16INK4A (Klaes, R., T. Friedrich, D. Spitkovsky, R. Ridder, W. Rudy, U. Petry, G. Dallenbach-Hellweg, D. Schmidt, and M. von Knebel Doeberitz. 2001. Int J Cancer 92:276-284; Sano, T., T. Oyama, K. Kashiwabara, T. Fukuda, and T. Nakajima. 1998. Am J Pathol 153:1741-8).

In some embodiments, the sample includes cells suspected of being from a HPV-associated tumor or precancerous lesion. In some embodiments, the sample includes cells from a routine test, e.g., a PAP smear, colonoscopy sample, or cheek swab. In some embodiments, the cells are in solution, e.g., in a liquid fixative such as Sure-Path (ethanol-based, TriPath Imaging) and Thin-Prep (methanol-based, Cytyc Corp). In some embodiments, the cells are smeared onto a slide, e.g., a conventional Pap smear (see, e.g., DeMay, M. (2007). Practical Principles of Cytopathology. Revised edition. Chicago, Ill.: American Society for Clinical Pathology Press). In some embodiments, the cells are in a pathology sample, e.g., a slice of tissue including cells known or suspected to be from an HPV-associated cancer or precancerous lesion. Such samples can be prepared using methods known in the art, e.g., fixation of a tissue sample and preparation of slices about 2-5, e.g., 3-4 mm thick.

Methods of Selecting a Treatment

In some embodiments, once it has been determined that a person has an HPV-associated precancerous lesion or cancer, or has an increased risk of developing an HPV-associated precancerous lesion or cancer, then a treatment, e.g., as known in the art or as described herein, can be administered. Knowing that a cancer is associated with HPV is particularly important since it allows optimal treatment decision-making.

Methods of treating cancers or precancerous lesions associated with HPV are known in the art. For example, methods of treating cervical lesions or cancers include surgical excision, e.g., loop electrosurgical excision procedure (LEEP), large loop excision of the transformation zone (LLETZ), cold-knife cone excision; cryotherapy (e.g., with nitrous oxide or carbon dioxide); chemotherapy; radiation therapy; or topical immunotherapies (e.g., with imiquimod or resiquimod).

Methods of Monitoring Treatment

The methods described herein can be used to monitor the efficacy of a treatment for HPV-associated cancers and precancerous lesions. The methods include obtaining a first sample from a subject who has an HPV-associated cancer and/or precancerous lesion at a first time point, and evaluating the presence and/or level of one or more of KDM6A, KDM6B, and/or H3K27me3 in the first sample; administering a treatment to the subject; obtaining a second sample from the subject at a subsequent time point, and evaluating the presence and/or level of one or more of KDM6A, KDM6B, and/or H3K27me3 in the second sample. A change in the presence and/or level of one or more of KDM6A, KDM6B, and/or H3K27me3 between the first and second samples indicates whether the treatment is effective. For example, an increase in levels of H3K27me3, and/or a decrease in a level of KDM6A and/or KDM6B, indicates that the treatment is effective, whereas no change or the opposite effect indicates that the treatment is not effective.

In some embodiments, the methods can be done on a surgical pathology tumor tissue specimen, to ensure that the entire cancerous or precancerous lesion has been removed. For example, during or after a surgical excision of a tumor or lesion, a pathological specimen can be evaluated using methods known in the art to determine whether the cells at the edges of the tissue removed are cancerous or precancerous. Similar to the methods of diagnosis, the methods can include evaluating the presence and/or level of one or more of KDM6A, KDM6B, and/or H3K27me3 in the first sample, and comparing the presence and/or level with one or more references, e.g., a control reference that represents a normal level of KDM6A, KDM6B, and/or H3K27me3, e.g., a level in n normal cell (e.g., from the same subject or a control subject), and/or a disease reference that represents a level of KDM6A, KDM6B, and/or H3K27me3 that is associated with cancer or a precancerous lesion, e.g., a level in a cell from an HPV-associated cancer or precancerous lesion. For example, a level of H3K27me3 in a cancerous or precancerous cell may be significantly (i.e., statistically significantly) reduced as compared to a normal control cell, e.g., substantially undetectable, and a level of KDM6A or KDM6B in a cancerous or precancerous cell may be significantly (i.e., statistically significantly) increased as compared to a normal control cell. The methods can include determining levels of KDM6A, KDM6B, and/or H3K27me3 protein or mRNA. In these methods, the presence of reduced levels of H3K27me3 and/or increased levels of KDM6A and/or KDM6B in all of the cells in the specimen could indicate that not all of the cancerous or precancerous cells had been removed, while the presence of surrounding cells with normal levels of KDM6A, KDM6B, and/or H3K27me3 could indicate that the entire lesion had been removed. In this embodiment, the surgical intervention can be repeated, or a non-surgical method can be applied, e.g., immunotherapy, chemotherapy, or radiation therapy.

Methods for detecting the presence of the KDM6A, KDM6B, and/or H3K27me3 protein or mRNA in a pathology specimen are known in the art, and can include immunological detection, e.g., immunohistochemistry or immunofluorescence. In some embodiments, additional methods of identifying HPV-infected cells are also used.

HPV Associated Cancers and Pre-Cancerous Lesions

The present methods can be used to diagnose and monitor any HPV-associated cancers or precancerous lesions. Such cancers and lesions arise in a number of tissues, including, but not limited to, cancers of the cervix, vulva, vagina, penis, anus, and some sites in the head and neck (oral cavity and oropharynx); breast; skin (non-melanoma, squamous cell carcinoma); prostate; and lung. See, e.g., De Vuyst H, Clifford G M, Nascimento M C, Madeleine M M, Franceschi S. International Journal of Cancer 2009; 124(7):1626-1636; Parkin D M, and Bray F. Vaccine 2006; 24 (suppl 3):S11-S25; Kreimer A R, Clifford G M, Boyle P, Franceschi S. Cancer Epidemiology, Biomarkers and Prevention 2005; 14(2):467-475; McNicol et al., J Clin Microbiol 1990 March; 28(3):409-12; Serth et al., Cancer Res 1999 Feb. 15; 59(4):823-5; Suzuki et al., Prostate 1996 May; 28(5):318-24; Klein et al., Lung Cancer. 2009 July; 65(1):13-8; Karagas et al., BMJ 2010; 341:c2986; Damin et al., Br Cancer Res Treat 84 (2): 131-137, (2004); Heng et al., Br J Cancer. 2009 Oct. 20; 101(8):1345-50; and Lawson et al., Br J Cancer. 2009 Oct. 20; 101(8):1351-6.

### EXAMPLES

The invention is further described in the following examples, which do not limit the scope of the invention described in the claims.

### Example 1

### H3K27me3 Levels are Reduced in HPV16 E7 Expressing Primary Human Epithelial Cells

HPV16 E7 binds to E2F6 containing PRCs and that detection of these complexes by immunofluorescence is reduced in HPV16 E7-expressing cells (McLaughlin-Drubin et al., (2008) J Virol 82(17):8695-8705). E2F6-containing PRCs bind to H3K27me3 transcriptional repressive marks. In order to determine if the reduced detection of E2F6-containing PRCs in HPV16 E7 expressing cells (McLaughlin-Drubin et al., (2008) J Virol 82(17):8695-8705) was accompanied by alterations in the H3K27me3 transcriptional repressive mark, the levels of mono-, di- and trimethylated H3K27 were compared by immunofluorescence in donor and passage-matched primary human foreskin keratinocyte (HFK) populations that were engineered to express HPV 16 E7 (HFK/E7) or were infected with an empty retroviral control vector.

Primary human foreskin keratinocytes (HFKs) and fibroblasts (HFFs) were isolated from anonymous newborn circumcisions and cultured as described previously (McLaughlin-Drubin et al., (2008) J Virol 82(17):8695-8705). Cells with stable expression of wild-type or mutant HPV16 E7 were generated by infecting with infection with pBABE, pBABE-16E7 (Piboonniyom et al. (2003) Cancer Res 63(2):476-483), and pBABE-E7 delD21-C24 (McLaughlin-Drubin et al., (2008) J Virol 82(17):8695-8705) that were produced as previously described (Piboonniyom et al. (2003) Cancer Res 63(2):476-483).

Immunofluorescence analysis of monolayer cells was performed as previously described (McLaughlin-Drubin et al., (2008) J Virol 82(17):8695-8705). Antibodies were used at the following dilutions: histone H3 (#9715, 1:50, Cell Signaling), H3K27me (#07-448, 1:500, Upstate), H3 K27me2 (#07-452, 1:500, Upstate), H3K27me3 (#07-4449, 1:500, Upstate), KDM6B (AP1022b, 1:500, Abgent), KDM6A (ab36938, 1:500, Abeam), and goat anti-rabbit Alexa Fluor 488 (1:1000, Invitrogen). Nuclei were counterstained with Hoechst 33258. Images were acquired using a Nikon Eclipse TE2000-E with a 60× objective and Metamorph 6.3r7 (Molecular Devices) software.

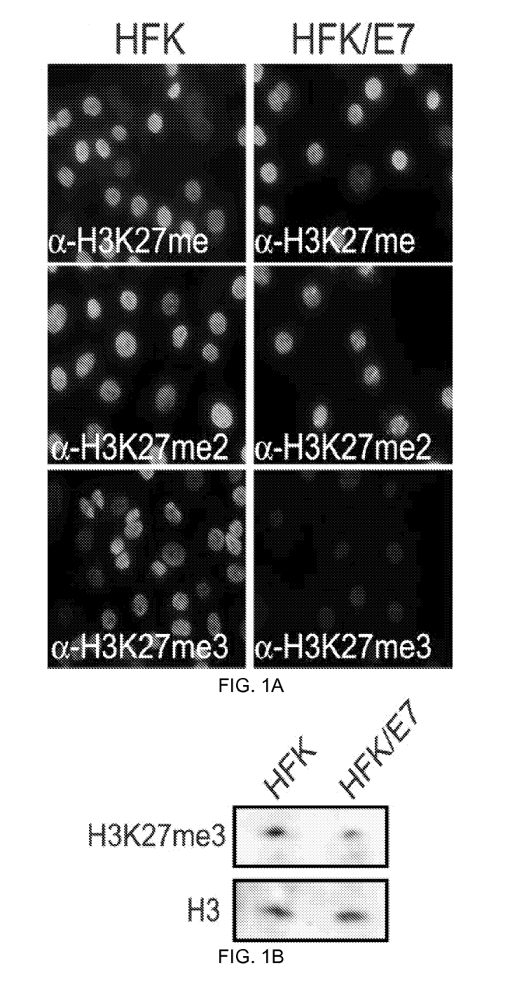

These experiments revealed a striking reduction in the intensity of H3K27me3 staining in HFK/E7 cells. In contrast there was no detectable reduction of H3K27me1 and H3K27me2 or total H3 staining in HFK/E7 as compared to control HFKs (FIG. 1A). A reduction of H3K27me3 levels in HFK/E7 was also detected by Western blotting (FIG. 1B).

### Example 2

### Expression of the H3K27 Specific Demethylases KDM6A and KDM6B is Increased in HPV16 E7 Expressing Primary Human Epithelial Cells

H3K27me3 repressive marks are placed by the histone methyltransferase containing PRC2 (Kuzmichev et al., (2002) Genes Dev 16(22):2893-2905; Cao et al. (2002) Science 298(5595):1039-1043; Czermin et al. (2002) Cell 111(2):185-196; Michel et al. (2002). Virology 294(1):47-59), and are removed by the histone demethylases KDM6A and KDM6B (Lee et al. (2007) Science 318(5849):447-450; De Santa et al. (2007) Cell 130(6):1083-1094; Lan et al. (2007) Nature 449(7163):689-694; Hong et al. (2007) Proc Natl Acad Sci USA 104(47):18439-18444). In order to determine if levels of the histone demethylases KDM6A and KDM6B were increased in HPV16 E7 expressing cells, immunofluorescence analyses were performed in HFK/E7 and donor and passage-matched control HFKs.

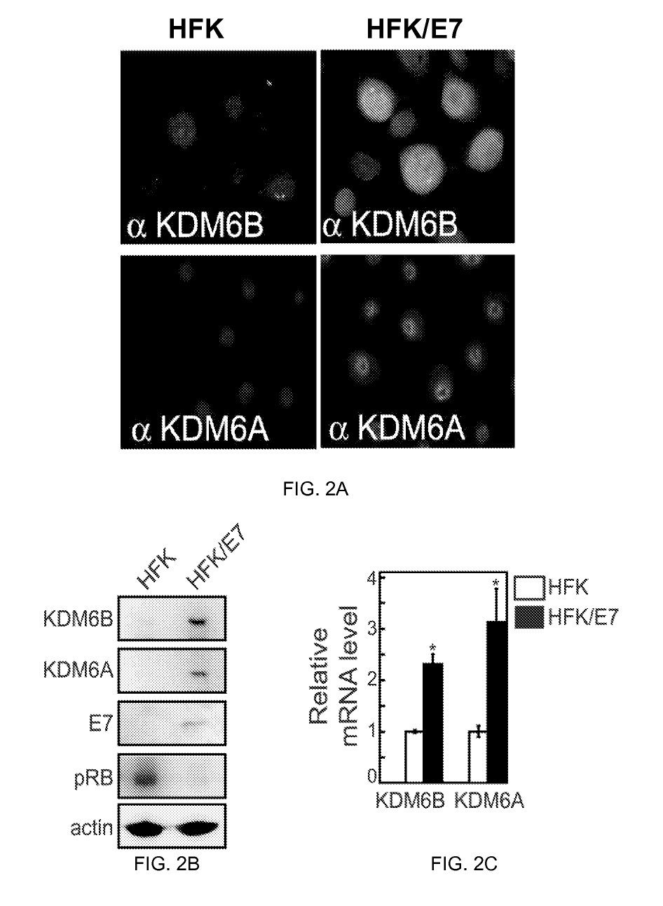

These experiments revealed a striking increase of KDM6A and KDM6B levels in HFK/E7s as compared to control HFKs (FIG. 2A).

Expression of KDM6A and KDM6B in HFK/E7 cells was also assayed by Western blot analysis. Cell lysates were prepared and processed as previously described (McLaughlin-Drubin et al., (2008) J Virol 82(17):8695-8705). Antibodies were used at the following dilutions: beta-actin (MAB1501, 1:1000, Chemicon), p14^{ARF} (sc-8613, 1:200, Santa Cruz), histone H3 (#9715, 1:1000, Cell Signaling), H3K27me3 (#9756, 1:1000, Cell Signaling), HPV16 E7 (mixture of 8C9, 1:150, Zymed/Invitrogen and ED17, 1:200, Santa Cruz Biotechnology), KDM6B (ab38113, 1:1000, Abeam), KDM6A (ab36938, 3 ug/ml, Abeam), p16^{INK4A} (sc-56330, 1:200, Santa Cruz), pRB (AB-5, 1:100, Oncogene Research), and HRP-conjugated secondary anti-rabbit (1:5000), anti-mouse (1:10,000), and anti-goat (1:5,000) (Amersham). Antigen/antibody complexes were visualized by enhanced chemiluminescence (Western Lightning Chemiluminescence Reagent Plus; PerkinElmer Life Sciences, Inc.) and exposed on Kodak BioMax XAR film or electronically acquired with a Kodak 4000R Image Station (Kodak) equipped with Kodak Imaging Software, version 4.0.

Increased expression of KDM6A and KDM6B in HFK/E7 cells was also detected by Western blot analysis (FIG. 2B).

Quantitative real time RT-PCR (qRT-PCR) experiments were performed as well to determine levels of KDM6A and KDM6B mRNA, as follows. Total RNA was extracted using the Total RNA Isolation Mini kit (Agilent). Quantitative reverse-transcription (RT) PCR analysis was performed using a 7300 real-time PCR system (Applied Biosystems) and the QuantiTect SYBR green RT-PCR kit (Qiagen). Primers for KDM6B and KDM6A were purchased from SABiosciences. Primers used for analysis of HOX gene expression (Takahashi et al. (2004) Exp Cell Res 293(1):144-153) are listed in Table 2. Cycling parameters were: 30 min at 50° C. for cDNA synthesis and 15 min at 95° C. for DNA strand denaturation, followed by 40 cycles for 15 s each at 94° C. (denaturation), 30 s at 55° C. (annealing), and 30 s at 72° C. (extension). Dissociation curve analysis (95° C. for 15 s, 60° C. for 15 s, and 95° C. for 15 s) was performed at the end to verify PCR product identity. Each RNA sample was tested in triplicate. Data were analyzed using the 2^{-ΔΔCT} method (Livak and Schmittgen (2001) Methods 25(4):402-408).

TABLE 2
Primers Used for Quantitative Real-Time RT-PCR
(5′ to 3′)

| | | | SEQ |
|---|---|---|---|
| | Forward/ | | ID |
| Gene | Reverse | Primer Sequence | NO: |
| HOXA1 | F | TCCTGGAATACCCCATACTTAGCA | 5 |
| HOXA1 | R | GCCGCCGCAACTGTTG | 6 |
| HOXA2 | F | ACAGCGAAGGGAAATGTAAAAGC | 7 |
| HOXA2 | R | GGGCCCCAGAGACGCTAA | 8 |
| HOXA3 | F | GCAAAAAGCGACCTACTACGA | 9 |
| HOXA3 | R | CGTCGGCGCCCAAAG | 10 |
| HOXA4 | F | TCCCCATCTGGACCATAATAGG | 11 |
| HOXA4 | R | GCAACCAGCACAGACTCTTAACC | 12 |
| HOXA5 | F | TCTCGTTGCCCTAATTCATCTTTT | 13 |
| HOXA5 | R | CATTCAGGACAAAGAGATGAACAGA | 14 |
| HOXA6 | F | CCCTCTACCAGGCTGGCTATG | 15 |
| HOXA6 | R | CAGGACCGAGTTGGACTGTTG | 16 |
| HOXA7 | F | CAAAATGCCGAGCCGACTT | 17 |
| HOXA7 | R | TAGCCGGACGCAAAGGG | 18 |
| HOXA9 | F | CCGAGAGGCAGGTCAAGATC | 19 |
| HOXA9 | R | AAATAAGCCCAAATGGCATCA | 20 |
| HOXA10 | F | ACAAGAAATGTCAGCCAGAAAGG | 21 |
| HOXA10 | R | GATGAGCGAGTCGACCAAAAA | 22 |
| HOXA11 | F | ACAGGCTTTCGACCAGTTTTTC | 23 |
| HOXA11 | R | CCTTCTCGGCGCTCTTGTC | 24 |
| HOXA13 | F | AAATGTACTGCCCCAAAGAGCA | 25 |
| HOXA13 | R | ATCCGAGGGATGGGAGACC | 26 |
| HOXB1 | F | CTCCTCTCCGAGGACAAGGAA | 27 |
| HOXB1 | R | CTGTCTTGGGTGGGTTTCTCTTAA | 28 |
| HOXB2 | F | TCCTTGGCCGTCTACTGGAA | 29 |
| HOXB2 | R | AGTGGATTAAACGCTAATTCAGTAATACC | 30 |
| HOXB3 | F | CCTGGCCTGAGAGGTTGCT | 31 |
| HOXB3 | R | TCCCGGGCGTGGAATT | 32 |
| HOXB4 | F | TTTTCAGCTTTGGCGAAGATG | 33 |
| HOXB4 | R | ACCGAGGCCCGTCTTCTC | 34 |
| HOXB5 | F | AGCGCCAATTTCACCGAA | 35 |
| HOXB5 | R | GGCTGCTTAGCTGGCTTGC | 36 |
| HOXB6 | F | AGCAGCCCCCGTTCCA | 37 |
| HOXB6 | R | AAAGGAGGAACTGTTGCACGAAT | 38 |
| HOXB7 | F | TCTTTAATGCTGTCTTTGTGGACTGT | 39 |
| HOXB7 | R | GAACACGCGAGTGGTAGGTTTT | 40 |
| HOXB8 | F | AACTCACTGTTCTCCAAATACAAAACC | 41 |
| HOXB8 | R | GACGGCCCGTGGTAGAACT | 42 |
| HOXB9 | F | AGGCCGTGCTGTCTAATCAAA | 43 |
| HOXB9 | R | CGAGCGTGCAGCCAGTT | 44 |
| HOXB13 | F | CCACTGGCTGCTGGACTGTT | 45 |
| HOXB13 | R | TATGACTGGGCCAGGTTCTTTG | 46 |
| HOXC4 | F | GGCAGCTACCCCGGGTACT | 47 |
| HOXC4 | R | TGTGAGTTATGTTTTATAACCTGGTAATGTC | 48 |
| HOXC5 | F | AGGTGCAGGCATCCAGGTACT | 49 |
| HOXC5 | R | GGGTTGGCAGCCATGTCTAC | 50 |
| HOXC6 | F | TCAAACGTGGACCTGAAAGTCA | 51 |
| HOXC6 | R | GGGAAAAGGGCCTGTAGACAA | 52 |
| HOXC8 | F | CGCACCACGTTCAAGACTTCT | 53 |
| HOXC8 | R | TAAGCGAGCACGGGTTCTG | 54 |
| HOXC9 | F | GGGCCCATCAGTAACTATTACGTG | 55 |
| HOXC9 | R | CGGTGGCCGGAAACCT | 56 |
| HOXC10 | F | CCTCGCAATGTAACTCCGAACT | 57 |
| HOXC10 | R | ACCCCGCAATTGAAGTCACT | 58 |
| HOXC11 | F | GTGAAGGGAAGTGTCTGATGCA | 59 |
| HOXC11 | R | AATCCGAGCAGCAAGACATTG | 60 |
| HOXC12 | F | TAATCTCCTGAATCCCGGGTTT | 61 |
| HOXC12 | R | TGGGTAGGACAGCGAAGGC | 62 |
| HOXC13 | F | AAGGTGGTCAGCAAATCGAAAG | 63 |
| HOXC13 | R | TGGTACAAAGCGGAGACATAAATAGA | 64 |
| HOXD1 | F | CGACCCCCATCCCTATCTAGAC | 65 |
| HOXD1 | R | TGGAACTCGGAAGCCAACTAAA | 66 |
| HOXD3 | F | CCATAAATCAGCCGCAAGGAT | 67 |
| HOXD3 | R | GATGGGTCTCAGACTTACCTTTGG | 68 |
| HOXD4 | F | GGCGGGATTCTCTCTCTAAGTATATTATA | 69 |
| HOXD4 | R | GAGCGGTGATTTTCATAAGTTTAATGA | 70 |
| HOXD8 | F | GATAACTTACAGAGACAGCCGATTTTTAC | 71 |
| HOXD8 | R | CCAATATTACCACTGGACGATTTACA | 72 |
| HOXD9 | F | CCAATATTACCACTGGACGATTTACA | 73 |
| HOXD9 | R | GTCGCCCTCATGGCCTATAA | 74 |
| HOXD10 | F | ATAAGCGCAACAAACTCATTTCG | 75 |
| HOXD10 | R | ATATCGAGGGACGGGAACCT | 76 |
| HOXD11 | F | CGGGCTGCGCCTACTATGT | 77 |
| HOXD11 | R | AGGACGACGGTTGGGAAAG | 78 |
| HOXD12 | F | TGTGTGAGCGCAGTCTCTACAGA | 79 |
| HOXD12 | R | CGGCCTCAGGTTGGAGAAG | 80 |
| HOXD13 | F | CTGGGCTACGGCTACCACTTC | 81 |
| HOXD13 | R | GCGATGACTTGAGCGCATT | 82 |
| GAPDH | F | GATTCCACCCATGCAAATCC | 83 |
| GAPDH | R | TGGGATTTCCATTGATGACAAG | 84 |

These experiments revealed that KDM6A and KDM6B mRNA levels are increased in HFK/E7 cells (FIG. 2C), and thus the mechanism of induction is at least in part transcriptional.

### Example 3

### HPV16 E7-Mediated Induction of the Cervical Cancer Biomarker p16^{INK4A} is Mediated by KDM6B

The cyclin-dependent kinase 4/6 inhibitor and tumor suppressor p16^{INK4A} is highly expressed in high-risk HPV-associated lesions and cancers and is an excellent biomarker for such malignancies (Sano et al., (1998) Am J Pathol 153(6):1741-1748; Klaes et al. (2001) Int J Cancer 92(2):276-284). KDM6B controls induction of p16^{INK4A} in response to oncogenic stress by ras/raf (Agger et al. (2009). Genes Dev 23(10):1171-1176; Barradas et al. (2009) Genes Dev 23(10):1177-1182). Given that HPV16 E7 induces expression of both p16^{INK4A} (Khleif et al. (1996) Proc Natl Acad Sci USA 93(9):4350-4354) and KDM6B (FIG. 2), it was investigated whether p16^{INK4A} positivity correlated with the loss of the H3K27me3 mark by immunofluorescence. Organotypic “raft” cultures prepared with primary HFKs and HPV16 genome immortalized HFKs were first analyzed.

Early passage HKFs immortalized by an integrated head-to-tail dimer of the HPV16 genome, Hkc/HPV16 (Pirisi et al., (1992) Cancer Res 52(1):187-193), were maintained in KSFM (Gibco/Invitrogen). Raft cultures were grown as previously described (Meyers et al., (1992) Science 257(5072):971-973) and were allowed to stratify for ten days.

Co-immunofluorescence microscopy of raft cultures was performed as previously described (Duensing et al., (2008) Virology 372(1):157-164). Antibodies were: H3K27me3 (#9756, 1:200, Cell Signaling), p16^{INK4A} (sc56330, 1:200, Santa Cruz), goat anti-rabbit Alexa Fluor 488 (1:1000, Invitrogen Molecular Probes), and goat anti-mouse Alexa Fluor 568 (1:1000, Invitrogen Molecular Probes). Nuclei were counterstained with Hoechst 33258. Images were acquired using an Axioplan 2 microscope (Zeiss) with a 63× objective and Axiovision 4.5 (Zeiss) software.

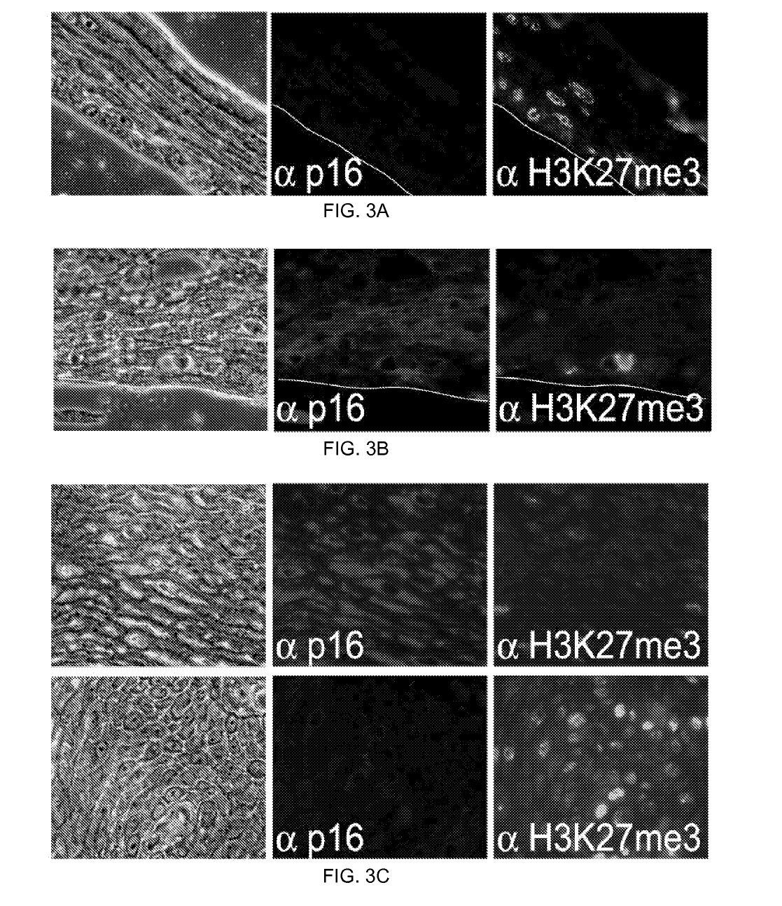

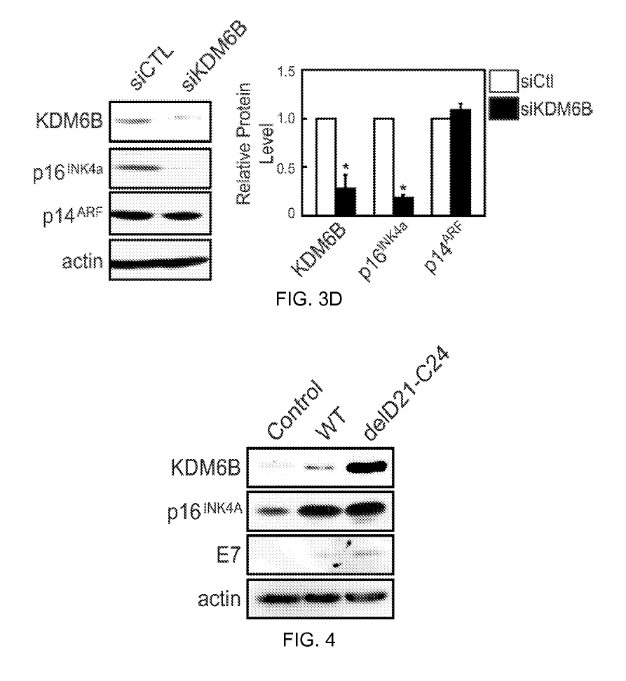

As expected, robust H3K27me3 and only weak p16^{INK4A} staining in HFK raft cultures (FIG. 3A) was detected. In contrast, most cells in the rafts prepared with HPV16 immortalized HKFs showed strong p16^{INK4A} and only weak H3K27me3 staining (FIG. 3B).

To determine whether p16^{INK4A} staining was also linked to a loss of the H3K27me3 signal in HPV16 associated clinical lesions, four HPV16 positive cervical intraepithelial neoplasia (CIN) specimens were next analyzed. Co-immunofluorescence microscopy of clinical specimens was performed as described above for raft cultures.

In areas with strong p16^{INK4A} staining little if any H3K27me3 staining was detected (FIG. 3C upper panels), whereas the presumably normal adjacent tissue on the same slide that exhibited weak p16^{INK4A} staining showed a robust H3K27me3 signal (FIG. 3C, lower panels).

To determine if increased expression of p16^{INK4A} is mechanistically linked to HPV16 E7-induced KDM6B expression, KDM6B was depleted in monolayer cultures of HPV16 immortalized HFKs by transfecting a pool of specific siRNA duplexes or control siRNA and KDM6B, p16^{INK4A}, and p14^{ARF} levels were analyzed by Western blotting.

1.75×10⁵Hkc/HPV16 cells were seeded onto six-well plates 1 day before transfection with 100 nM KDM6B specific ON-TARGET plus SMARTpool (L-023013-01, Thermo Scientific Dharmacon) or ON-TARGET plus Non-Targeting Pool (D-001810-10, Thermo Scientific Dharmacon) using Lipofectamine 2000 (Invitrogen).

The results indicated that KDM6B depletion caused a decrease in p16^{INK4A} levels as compared to control siRNA transfected cells, while p14^{ARF} levels were unchanged (FIG. 3D).

### Example 4

### HPV16 E7-Mediated Induction of KDM6B and its Target p16^{INK4A} is not Strictly Dependent on pRB Inactivation

To determine whether induction of KDM6B expression may represent a consequence of “oncogenic stress” in response to HPV16 E7 mediated pRB inactivation, expression of KDM6B and its target p16^{INK4A} were compared by Western blotting in a set of donor and passage matched primary human fibroblasts with expression of wild type HPV16 E7 or the pRB-binding and degradation-deficient HPV16 E7 delD21-C24 mutant as well as control vector transduced cells.

Expression of the HPV16 E7 delD21-C24 mutant robustly increased KDM6B and p16^{INK4A} levels (FIG. 4). Similar results were obtained by transient transfection experiments in U2OS cells. Hence, upregulation of KDM6B and its transcriptional target p16^{INK4A} by HPV16 E7 are not dependent on pRB inactivation.

### Example 5

### Deregulated HOX Gene Expression in HPV16 E7 Expressing Primary Epithelial Cells

HOX A-D loci are well-established transcriptional targets of PRCs (Bracken et al., (2006) Genes Dev 20(9):1123-1136). To determine whether the HPV16 E7 mediated increases in KDM6A and KDM6B expression causes changes in HOX gene expression, expression of the 39 HOX A-D genes in HFK/E7 cells was compared to donor and passage matched control HFKs by qRT PCR.

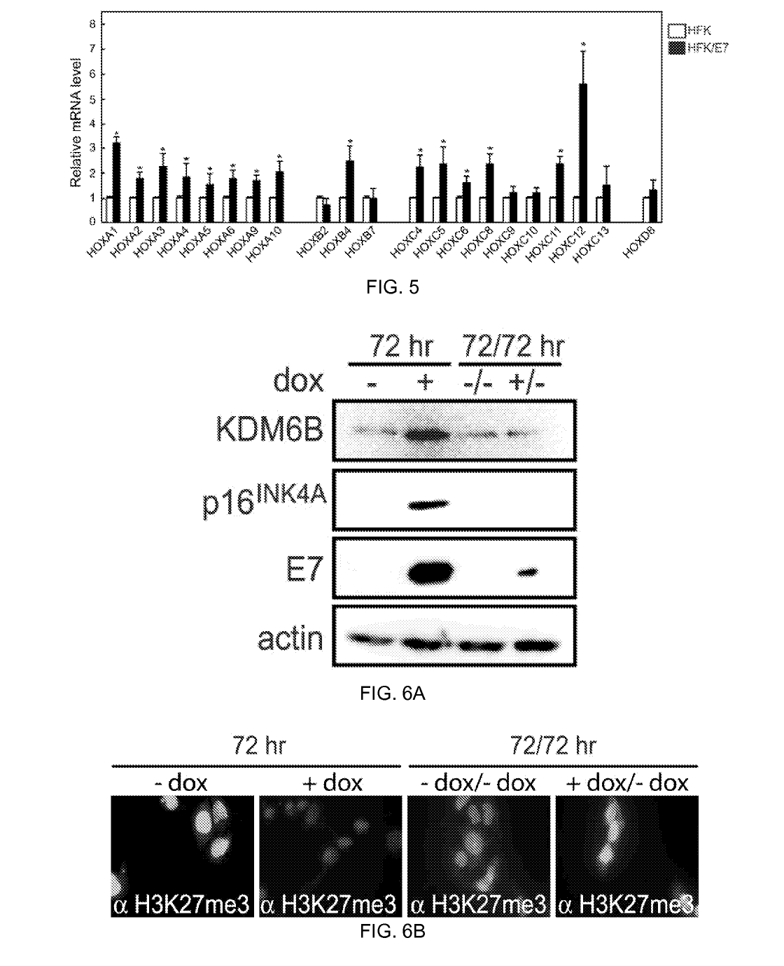

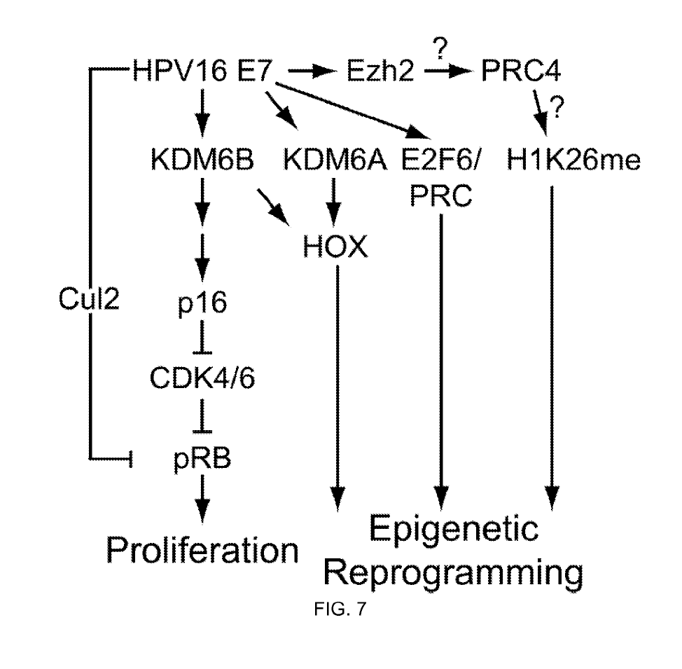

These experiments revealed that mRNA levels of a number of HOX genes are significantly increased in HFK/E7 cells (FIG. 5). Hence, HPV16 E7 expression causes epigenetic reprogramming of primary human epithelial cells.

### Example 6

### HPV16 E7 Mediated Induction of KDM6B and p16^{INK4A} Expression is Reversible

To determine if induction of KDM6B by HPV16 E7 is reversible, U2OS cells with doxycycline-inducible expression of HPV16 E7 were used.

Increases in KDM6B and p16^{INK4A} expression (FIG. 6A), with concomitant decreases in the H3K27me3 mark (FIG. 6B), were observed upon doxycycline induced HPV16 E7 expression. These changes were abolished when HPV16 E7 expression was extinguished by removal of doxycycline (FIG. 6). Hence, the observed induction of KDM6B expression, associated decreases in H3K27me3 levels, and induction of p16^{INK4A} expression are a direct consequence of HPV16 E7 expression, and these alterations are reversible upon silencing of HPV16 E7 expression.

These results indicate that expression levels of H3k27me3 and KDM6A/B can be used to monitor efficacy of treatment in patients with cancers associate with HPV infection.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of diagnosing a human papilloma virus (HPV)-associated cancer or precancerous lesion in a subject, the method comprising:
obtaining a sample comprising a cell from the subject;
evaluating the presence and/or level of one or more biomarkers selected from the group consisting of lysine (K)-specific demethylase 6A (KDM6A), KDM6B, or Histone H3 Lys27 trimethyl (H3K27me3) in the sample;
wherein the presence and/or level of the one or more biomarkers indicates whether the subject has an HPV-associated cancer or precancerous lesion.

2. The method of claim 1, further comprising comparing the level of the biomarker with a reference level, wherein the comparison of the level of the biomarker with the reference indicates whether the subject has an HPV-associated cancer or precancerous lesion.

3. The method of claim 2, wherein the reference is a control reference that represents a level of KDM6A, KDM6B, and/or H3K27me3 in a normal cell, or a disease reference that represents a level of KDM6A, KDM6B, and/or H3K27me3 in a cell from an HPV-associated cancer or precancerous lesion.

4. The method of claim 1, wherein the level of H3K27me3 is determined, and the level of H3K27me3 is reduced as compared to a level in a normal control cell, then the subject is diagnosed with an HPV-associated cancerous or precancerous cell.

5. The method of claim 1, wherein the presence of H3K27me3 is determined, and the absence of detectable levels of H3k27me3 indicates that the subject has an HPV-associated cancer or precancerous lesion.

6. The method of claim 1, wherein:
the level of H3K27me3 is determined,
a level of total histone3 (H3) is determined, and
a ratio of H3K27me3 to total H3 is calculated, wherein the ratio of H3K27me3 to total H3 in the cell indicates whether the cell is from an HPV-associated cancer or precancerous lesion.

7. The method of claim 1, wherein a level of KDM6A or KDM6B is determined, and the presence of a level of KDM6A or KDM6B that is be significantly increased as compared to a normal control cell indicates that the subject has an HPV-associated cancer or precancerous lesion. The methods can include determining levels of

8. The method of claim 1, further comprising selecting and/or administering a treatment for an HPV-associated cancer or precancerous lesion to the subject.

9. A method of monitoring the efficacy of a treatment for an HPV-associated cancer or precancerous lesion, the method comprising:
obtaining a first sample from a subject who has an HPV-associated cancer and/or precancerous lesion at a first time point, and evaluating the presence and/or level of one or more of KDM6A, KDM6B, and/or H3K27me3 in the first sample;
administering a treatment to the subject;
obtaining a second sample from the subject at a subsequent time point, and evaluating the presence and/or level of one or more of KDM6A, KDM6B, and/or H3K27me3 in the second sample;
comparing the level of the KDM6A, KDM6B, and/or H3K27me3 in the first and second sample, wherein a decrease in the level of KDM6A, KDM6B, and/or an increase in the level of H3K27me3 between the first and second samples indicates that the treatment is effective.

10. The method of claim 1, comprising determining a level of KDM6A, KDM6B, and/or H3K27me3 protein or mRNA.

11. The method of claim 1, wherein the subject is a mammal.

12. The method of claim 1, wherein the HPV-associated cancer or precancerous lesion is in a tissue selected from the group consisting of cervix, vulva, vagina, penis, anus, oral cavity, oropharynx, breast, skin, prostate, and lung.

13. The method of claim 1, further comprising determining a level of an additional marker of HPV-associated cancer.

14. The method of claim 13, wherein the additional marker is p16^{INK4A}.

15. The method of claim 9, comprising determining a level of KDM6A, KDM6B, and/or H3K27me3 protein or mRNA.

16. The method of claim 9, wherein the subject is a mammal.

17. The method of claim 9, wherein the HPV-associated cancer or precancerous lesion is in a tissue selected from the group consisting of cervix, vulva, vagina, penis, anus, oral cavity, oropharynx, breast, skin, prostate, and lung.

18. The method of claim 9, further comprising determining a level of an additional marker of HPV-associated cancer.

19. The method of claim 18, wherein the additional marker is p16^{INK4A}.
